(19) United States
(12) Patent Application Publication
(10) Pub. No.: US 2025/0248675 A1
(43) Pub. Date: Aug. 7, 2025

(54) REDUCTION OF ARTIFACTS IN SPECTRAL COMPUTED TOMOGRAPHY IMAGE DATA

(71) Applicant: Siemens Healthineers AG, Forchheim (DE)

(72) Inventors: Johan SUNNEGAARDH, Adelsdorf (DE); Karl STIERSTORFER, Erlangen (DE); Martin PETERSILKA, Adelsdorf (DE); Julien ERATH, Erlangen (DE)

(73) Assignee: Siemens Healthineers AG, Forchheim (DE)

(21) Appl. No.: 19/032,163

(22) Filed: Jan. 20, 2025

(30) Foreign Application Priority Data
Feb. 6, 2024 (EP) 24156011.9

(51) Int. Cl. A61B 6/00; A61B 6/03; G06T 11/00

(52) U.S. Cl. CPC A61B 6/5282; A61B 6/03; A61B 6/482; G06T 11/008; G06T 2210/41; G06T 2211/452

(57) ABSTRACT

A method comprises: receiving computed tomography measurement data with sets of measurement values, wherein a set corresponds to a detector element readout of the measurement data including measurement values from at least two different x-ray spectra; determining an error estimate per set of measurement values; defining a replacement reference value per set of measurement values; applying a function that maps the measurement values to corrected values, the corrected values being between the mapped measurement value and the replacement reference value of the respective set, such that for larger errors the corrected value is closer to or at the replacement reference value and for smaller errors the corrected value is closer to or at the measurement value; and reconstructing computed tomography image data based on the corrected values.

### CROSS-REFERENCE TO RELATED APPLICATION(S)

The present application claims priority under 35 U.S.C. § 119 to European Patent Application No. 24156011.9, filed Feb. 6, 2024, the entire contents of which is incorporated herein by reference.

### FIELD

One or more example embodiments of the present invention relate to a method for the reduction of artifacts in spectral computed tomography image data, a method for providing spectral computed tomography image data, a computer program, a non-transitory computer-readable storage medium, and/or a computed tomography system.

### BACKGROUND

Independent of the grammatical term usage, individuals with male, female or other gender identities are included within the term.

In spectral computed tomography (CT), x-ray attenuation measurements from multiple x-ray spectra, e.g. from two different x-ray spectra, are acquired from an object of interest. For example, to generate the measurements with different x-ray spectra, multiple measurements with different x-ray source voltages or beam filtrations may be used. Additionally or alternative, energy-resolved detectors, such as photon counting or dual layer detectors, may be deployed. The measured x-ray attenuation depends on the material that is scanned and the applied x-ray spectrum. Therefore, differences between attenuation measurements acquired for different x-ray spectra may be used to differentiate between materials that have the same x-ray attenuation for a single x-ray spectrum.

The mathematical problem of decomposing measured x-ray attenuations for different spectra into a basis suitable for clinical applications, e.g., water and iodine, is called material decomposition. Errors that occur prior to material decomposition may be caused by physical effects such as scattered radiation, extra-focal radiation, or very low signal counts in low dose situations. Errors may also be caused by imperfect data correction steps intended to correct for physical effects occurring during data acquisition. Typically, material decomposition does not reduce these errors. On the contrary, material decomposition tends to even increase the errors. Hence, any errors in the data or images prior to material decomposition get amplified and may result in image artifacts. Image artifacts may, for example, comprise streak artifacts, cupping artifacts and degraded contrast-to-noise ratio of the images.

In state-of-the-art CT systems, to minimize data errors prior to material decomposition, typically much effort is put in calibration and computational steps to correct for physical effects as accurately as possible.

Furthermore, the data acquisition modes available for spectral acquisition may be restricted in order to reduce the physical effects causing image artifacts. For example, the x-ray beam collimation may be reduced in order to reduce scattered radiation. However, this can have a direct negative implication on the clinical application: a smaller collimation typically results in a longer examination time and higher risk of motion artifacts. A longer examination time may also increase the amount of contrast agent required for the examination.

### SUMMARY

It is an object of one or more example embodiments of the present invention to provide a mechanism and/or means to reduce the occurrence of image artifacts.

At least this object is met or exceeded by methods, computer programs, non-transitory computer-readable storage mediums, and/or computed tomography systems as claimed. Further advantages and features result from dependent claims, the description and the attached figures.

According to a first aspect of embodiments of the present invention, a method for the reduction of artifacts in spectral computed tomography image data is provided. The method comprises:
(a) receiving computed tomography measurement data with sets of measurement values, wherein one set corresponds to one detector element readout of the measurement data comprising measurement values from at least two different x-ray spectra;
(b) determining and/or receiving an error estimate per set of measurement values;
(c) defining one replacement reference value per set of measurement values for at least some of the sets;
(d) applying a function that maps the measurement values to corrected values, the corrected values being in between the mapped measurement value and the replacement reference value of the respective set, the distance of the corrected value to each depending on the error estimate such that for larger errors the corrected value is closer to or at the replacement reference value and for smaller errors the corrected value is closer to or at the measurement value;
(e) reconstructing computed tomography image data based on the corrected values.

Advantageously, the method may allow to reduce artifacts, in particular due to the step for mapping measurement values to the corrected values. For example, artifacts comprising streak artifacts may thus be reduced. Thus, for example, more challenging modes, such as dual source acquisition or large collimation may be enabled without introducing prohibitively strong artifacts. The method may be particularly advantageous, if data errors occur in relatively small portions of the whole measured data. Hence, preferably, only some data, in particular a minority of data, comprise substantial errors. For example, the method may be particularly advantageous, if less than 10% of the measured data have significant data errors. Small portions of data may be so erroneous that, without a correction, an attempt to perform a material decomposition may likely only add artifacts to the image. It has been found, that even only very few measurement values that are affected by errors, may have a large influence on artifacts on the image over a large part of the image. In particular even far less than 10% of the data may be affected by errors, but still lead to significant artifacts on the image. For example, only measurement data associated with particular x-rays from a particular direction may be affected. Advantageously, this effect of few erroneous measurement values having a negative impact on the whole image may advantageously be avoided by this method. On the other hand, these very few data, due to their relatively low number, may be of relatively minor importance for the spectral decomposition itself, such that still sufficient spectral information may remain.

The term “computed tomography (CT) measurement data” may be understood broadly in the context of this invention. It generally describes data acquired with a computed tomography system comprising measurement values. In the context of this invention, the x-ray intensity measurement data may in particular be x-ray intensity measurement data and/or attenuation measurement data. For example, attenuation measurement data may be derived from x-ray intensity measurement by applying a negative algorithm, such μ=−log^{I}/I₀ with μ being the attenuation, I being the x-ray intensity and I₀ being the x-ray intensity that would be measured without an attenuating object. The method according to embodiments of the present invention is generally applicable to both, x-ray intensity measurement data and attenuation measurement data. The measurement values may be acquired by sending x-rays from an x-ray source, in particular an x-ray tube, at least one x-ray detector. For example, the at least one x-ray detector may comprise a row and/or an array of x-ray detectors. Typically, an object is placed between the x-ray source and the at least one x-ray detector, such that the x-rays are attenuated depending on the material, e.g. tissue material, of the object before reaching the detector. For example, the object may be part of a patient. Typically, the x-rays generated by the x-ray source comprise a spectrum of x-ray frequencies. Depending on the specific spectrum of x-rays and the material that is in the pathway of the x-rays, the attenuation of the x-rays may differ. Recording the x-ray attenuation for different spectra may thus help to distinguish between different materials.

In the context of this invention, the measurement values may be put together in sets of measurement values. One set corresponds to one detector element readout of the measurement data comprising measurement values from at least two different x-ray spectra. The term “detector element readout” is to be understood broadly in the context of this invention. It generally describes a part of the at least one x-ray detector of the CT system. A detector element may correspond to a group of detector pixels or, preferably, to a single detector pixel. The detector element readout may, in particular, be a detector pixel readout. For example, the at least one x-ray detector may be an energy resolved detector, such as a photon counting detector or a dual layer detector. At least one energy resolved detector may be used to detect different x-ray spectra. Additionally or alternatively, for example, different x-ray spectra may be generated by applying different x-ray source voltages and/or different beam filtrations. Due to the sets comprising measurement values from at least two different x-ray spectra, a reconstruction of spectral data, allowing a better distinction between some materials may be enabled. For example, receiving the computed tomography measurement data may comprise retrieving the data from a database and/or directly receiving the data during a CT scan.

For the sets of measurement values an error estimate is determined and/or received. In particular one error estimate per set of measurement values may be determined and/or received. Hence, every detector element readout may be associated with an error. For example, the error estimate may be determined and/or chosen such that the error estimate of one of the measurement values of a set is chosen that yields the highest error estimate out of the error estimates for the measurement values of one set. For example, the error estimate may be stored in a database, such as in a database together with the measurement data. In this case, the error may be retrieved together with the measurement data. For example, the error estimate may be estimated by an external processor and provided for the method. Alternatively, the error estimate may be determined during this method itself. The error estimate may be an error estimate on an error scale that defines the severity of the error. The error estimate may be an estimation that defines whether there is an error or whether there is no error. The error estimate may be defined according to an error metric. A substantial error may be defined to exist, if the error metric is above a defined threshold.

For at least some of the sets of measurement values, a replacement reference value is defined. The replacement reference value may be defined based on the values of the measurement values of the set. Optionally, for some of the sets, in particular sets that have an error estimate that is below a threshold or that are determined not to have a substantial error, the defining of a replacement reference value may be omitted. Accordingly, the following step of applying a function may, optionally, only be applied to sets for which a replacement reference value has been defined. The replacement reference value may serve as a reference for the mapping of the measurement values to the corrected values.

The function that maps the measurement values to corrected values is in particular applied based on the error estimate and the replacement reference value. The output of the function is in particular a respective corrected value. The corrected values are in between the mapped measurement values and the replacement reference values. In the context of this function “in between” may also comprise the end values of the described range, i.e. the replacement reference values and the measurement value. In other words, at least in some cases, the corrected value may be the replacement reference value or the actual measurement value. In particular when the estimated error is low or substantially non-existent, the corrected value may be set to be the measurement value. On the other hand, a relatively large error may lead to a correction of the measurement value to a different corrected value. In particular for sets with relatively large errors, each measurement value of the set may be mapped to a corrected value, such that the corrected values are the same or more similar than the original measurement values for the whole set. Since the spectral decomposition is typically determined based on a (relatively small) difference of the measurement values from different spectra, the difference typically is enhanced in the reconstructed image data, e.g. by multiplication with a relatively large factor. Thus, errors in the measurement values can lead to large artifacts due to the errors being larger or much larger than the difference caused by the application of different spectra. Advantageously, the method may thus allow to reduce the effect of these errors. On the other hand, since, the mapping is dependant on the errors, a spectral reconstruction of areas associated with reliable data can still be performed. While some true spectral content of data associated with high errors may be reduced this way, it has been found that, for the end result, it is typically better to avoid an introduction of artifacts than to try to use the spectral information of erroneous measurement values. With large errors any true spectral content would typically be masked by artifacts.

The corrected values are then used as basis for reconstructing computed tomography image data. In particular spectral computed tomography image data may be reconstructed. The method, according to embodiments of the present invention, may thus provide a relatively simple mechanism and/or means of treating unreliable data with less weight during a spectral reconstruction, while still allowing for a spectral reconstruction of areas associated by reliable data.

According to an embodiment, the replacement reference value is defined to be in the order of magnitude of the measurement values of the respective set of measurement values. The order of magnitude may, for example be, defined to be a decimal or binary order of magnitude. Hence, for example, the order of magnitude may correspond to the power of 10 of the respective value. Advantageously, using the same order of magnitude may be a simple mechanism and/or means of ensuring that the corrected value, being in between the replacement reference value and the measurement value, will also be of the same order of magnitude as the measurement values. Thus, the corrected value does not deviate too much from the measurement values.

According to an embodiment, the replacement reference value is defined such that it is a linear combination of the measurement values of one set of measurement values. Using a linear combination may provide a relatively simple measure to ensure that the replacement reference value corresponds well to the measurement values of the set.

According to an embodiment, the coefficients of the linear combination are the same within each mapped set. Using the same coefficients may be a particularly simple way of determining the replacement reference values, without the need to determine coefficients for each set individually. Additionally or alternatively, the sum of coefficients of the linear combination is essentially 1. In other words, the linear combination may be a convex combination. A convex combination may allow to achieve a simple mechanism and/or means to ensure that the order of magnitude of the in the order of magnitude of the measurement values of the respective set of measurement values.

For example, a corresponding linear combination may be defined as explained in the following. The list {S₁, S₂, . . . , S_{N}} may represent attenuation or x-ray intensity measurements from N different spectra for a single detector element readout, in particular detector pixel readout. Hence, {S₁, S₂, . . . , S_{N}} are measurement values from a set. For example, in the case of a dual energy CT, the list may be of the form {S₁, S₂}. A corresponding error estimate may be defined as w. The error may be defined in a range of errors, different parts of the range corresponding to different severities of the error. For example, the error estimate may be in the range [0,1], 0 meaning no error and 1 meaning an error so high that the respective set is unsuitable for spectral processing. However, generally, other measures of the error may be applied as well. The replacement reference value S* may than be defined to be a linear combination of the measurement values from the list of measurement values of the set {S₁, S₂, . . . , S_{N}}. S* may in particular be a convex combination of the measurement values {S₁, S₂, . . . , S_{N}}. Hence, for example, S* may have the form

S*=Σₖ₌₁^{N}cₖSₖ,
where cₖ≥0, k=1, . . . , N and Σₖ₌₁^{N}cₖ=1.

According to an embodiment, the function that maps the measurement values to corrected values is a monotonic function. Applying a monotonic function may be a particularly useful way of selectively reducing spectral content for sets of measurement values with high errors. Preferably, the function is a smooth monotonic function. For example, measurement values Sₖ may be mapped by a smooth monotonic function that maps the measurement values Sₖ to a value between Sₖ and S*, as defined above. For example, the function may map the measurement values Sₖ to the corrected values Sₖ^{corr} in the form of

Sₖ^{corr}=ƒ(w;Sₖ),
where ƒ(0; Sₖ)=Sₖ and ƒ(1; Sₖ)=S*. Hence, in this example and for an error w=1 the functions may replace the measurement values Sₖ with the replacement reference value S*, thus setting the corrected values Sₖ^{corr} to be the respective replacement reference value S*. On the other hand, in this example, the measurements values Sₖ are mapped to a value closer to S* for high errors w, and closer to Sₖ for low errors w. Hence, for detector element measurements associated with a high error estimate, the difference between the corrected values Sₖ^{corr} of a set is reduced compared to the difference of the measurement values of the set {S₁, S₂, . . . , S_{N}}. This reduction, or even complete omission of the difference, leads to a smaller impact of the associated error of the measurement values on the reconstructed image.

According to an embodiment, the replacement reference value is defined to be one of the measurement values of the respective set of measurement values. Using one of the measurement values as replacement reference value may be a particularly simple mechanism and/or means of generating the replacement reference value. This embodiment may correspond to a linear combination where one coefficient is 1 and the other coefficients are set to be zero. The replacement reference value may be chosen such that the most reliable of the measurement values is chosen. For example, the most reliable of the measurement values may be chosen based on an estimated error. For example, the measurement value corresponding to a spectrum that in itself has fewer artifacts or provides better statistical properties may be chosen. Alternatively measurement value to be used as replacement reference value may be chosen randomly. This may be beneficial in being particularly simple, while the main advantage of avoiding an increase in error due to calculating a difference between measurement values of the set can still be achieved. For example, in the case of large errors, all the measurement values may be set to take the value of one of the measurement values of the set. Thus, effectively, certain unreliable data, i.e. having a large error estimate, may be treated as non-spectral, since the corrected values all have the same value and thus no spectral difference.

According to an embodiment, the error estimate is based on determining the error of data that is erroneous due to scattered radiation. The error due to scattered radiation may be estimated by a scatter correction algorithm. For example, errors may be caused due to forward scattering or cross scattering reaching the x-ray detector. In a setup with two x-ray sources and detectors at the opposite side of each x-ray source, due to cross scattering, x-rays may be detected by the wrong detector, i.e. the detector that is not opposite the respective x-ray source. Forward scattering is typically known to be present in all x-ray devices. Scattered radiation may lead to additional signals in detector elements.

According to an embodiment, the error estimate is determined based on a ratio, optionally a maximum ratio, between an estimated scatter signal and a total signal strength of the respective measurement values. A maximum error may be understood such, that, for the error estimation, the error of that measurement value of the set is used that yields the highest error estimate. Alternatively other ratios, besides the maximum ratio, may be used. For example, a minimum ratio may be applied. For example, a ratio corresponding to one particular energy and/or one particular spectrum in the set may be applied. For example, two spectra may be acquired. In some cases, it may be advantageous, to reconstruct an image corresponding almost fully to only one of the spectra. In this case, it may be preferable to use the scatter/total ratio corresponding to this spectrum only. A scatter signal can be understood to describe the part of the total signal that is due to scattered x-ray radiation. For example, the scattered x-ray radiation may comprise forward scattering and/or cross scattering. A corresponding error estimate is typically particularly high where the total signal is low and the scattered radiation is high. For example, this is often observed for x-rays passing through both shoulder blades of a patient. Attempting to use affected measurements spectrally may lead to severe streak artifacts along the x-rays passing through both shoulder blades. By applying the method for the reduction of artifacts, the severity of such artifacts can be reduced while leaving areas with lower errors unaffected.

For example, a scattered radiation Sₖ^{sc}≥0 may be estimated. The ratio between estimated scatter and total signal may be defined by Sₖ^{sc}/Sₖ, k=1, . . . , N, N being the number of spectra. This definition may be used to construct an error estimate

$$w = {max}_{k} min ( 1 , {S}_{k}^{s c} / {S}_{k} ) .$$

Hence, a maximum error is determined by choosing the k for which the error is maximal, i.e. hence the error is determined for the measurement value of the set, for which the error is maximal.

Methods for the estimation of scattered radiation are generally known in the state of the art. For example, a scattering may be estimated by model-based profiles and/or by direct measurement of the scattered radiation. An estimation based on model-based profiles may comprise a model of the scatter process which is used to estimate the scattering based on the measured signal. A direct measurement may, for example, be performed by dedicated scatter sensors that are used to measure the scattered radiation. Examples for a model-based estimation and for direct measurement of scattered radiation have been published in the state of the art, such as by Petersilka et al. in “Strategies for scatter correction in dual source CT”, Med. Phys. 37 (11), November 2010, 5971-5992.

According to an embodiment, the error estimate is determined based on data errors in a low signal domain, such that a high error is assumed for low detector signals. For example, for low detector signals, statistical errors may render measurements too unreliable to be used for spectral reconstruction. An error estimate may be high for low signals and low for high signals. For example, a particularly big patient may lead to a low signal domain due to a long pathway of the x-rays through the patient's body. For example, some x-ray pathways through an examined object may lead to a complete or nearly complete attenuation of the x-rays in this direction. For example, if the number of photons pear measurement value is lower than a threshold the measurement value may be marked as erroneous. For example, measurement values based on a signal that is lower than a noise level may be determined to be unusable. For example, an error estimate for data errors in a low signal domain may be constructed via the form

$$w = {max}_{k} {cos}^{2} ( \frac{π}{2} max ( 0 , min ( 1 , \frac{{S}_{k} - {S}_{k}^{L}}{{S}_{k}^{H} - {S}_{k}^{L}} ) ) ) ,$$

where the parameters Sₖ^{L} and Sₖ^{H}, k=1, . . . , N define the signal level range where measurements are to be considered as unreliable, i.e. erroneous.

According to an embodiment, the mapping is defined according to at least two different error ranges of the estimate, such that for each error range a different partial mapping function is applied. For example, one error range may correspond to the measurement values being not being changed during the mapping, i.e. corrected values being the same as the measurement values, and a second error range may correspond the measurement values being mapped to be closer or at the corresponding replacement reference value.

According to an embodiment, the error estimate comprises at least three error ranges according to which the mapping is applied, the error ranges comprising a first error range of low errors for which the corrected values are set to be the measurement value, a second error range of medium errors for which the measurement values are mapped such that the corrected values are between the corresponding measurement value and the respective replacement reference value, and a third error range of high errors for which the corrected values are set to be the replacement reference value of their respective set. The first error range may be below a first error threshold. The third error range may be above a second error threshold. The second error range may be between the first and the second error threshold. In particular, the first error range may correspond to errors that are lower than the errors of the second error range of and the second error range may correspond to errors that are lower than the errors of the third error range. The first error range and the third error range may each be directly adjacent to the second error range. Optionally, there may be more than three error ranges. For example, there may be different types of medium error ranges, such that a different mapping is applied for the different types of medium error ranges.

The embodiments described herein may be combined with each other unless indicated otherwise. For example, the replacement reference values may be defined via a linear combination and, at the same time, the mapping may be defined via error ranges.

According to a further aspect of embodiments of the present invention, a method for providing spectral computed tomography image data is provided. The method comprises
acquiring measurement data by performing a spectral computed tomography scan,
performing the method for the reduction of artifacts in spectral computed tomography image data as described herein.

All features and advantages of the method for the reduction of artifacts may be adapted to the method for providing spectral computed tomography image data and vice versa.

According to a further aspect of embodiments of the present invention, a computer program comprising instructions which, when the program is executed by a computer, cause the computer to carry out the method as described herein, in particular the method for the reduction of artifacts in spectral computed tomography image data. All features and advantages of the method for the reduction of artifacts and of the method for providing spectral computed tomography image data may be adapted to the computer program and vice versa.

According to a further aspect of embodiments of the present invention, a computer-readable storage medium, in particular non-transient or non-transitory computer-readable storage medium, comprising instructions which, when executed by a computer, cause the computer to carry out the method as described herein, in particular the method for the reduction of artifacts in spectral computed tomography image data. All features and advantages of the method for the reduction of artifacts, of the method for providing spectral computed tomography image data, and of the computer program may be adapted to the computer-readable storage medium and vice versa. The storage medium may be any computer-readable storage medium. For example, the storage medium may be an optical storage medium or a solid-state storage medium. The storage medium may be a hard-drive, a solid-state-disk a flash storage, an online server etc.

According to a further aspect of embodiments of the present invention, a computed tomography system comprising a processing unit that is configured to carry out any of the methods as described herein, is provided. All features and advantages of the method for the reduction of artifacts, of the method for providing spectral computed tomography image data, of the computer program, and of the computer-readable storage medium may be adapted to the computed tomography system and vice versa.

### BRIEF DESCRIPTION OF THE DRAWINGS

The accompanying drawings illustrate various exemplary embodiments and methods of various aspects of the present invention.

FIG. 1 shows a flow diagram of a method for the reduction of artifacts in spectral computed tomography image data according to an embodiment of the present invention;

FIG. 2 shows a mapping function according to an embodiment of the present invention;

FIG. 3 shows a reconstructed spectral computed tomography image, where measurement values have not been corrected, according to embodiments of the present invention;

FIG. 4 shows a reconstructed spectral computed tomography image based on corrected values according to embodiments of the present invention;

FIG. 5 shows a flow diagram of a method for providing spectral computed tomography image data according to an embodiment of the present invention; and

FIG. 6 shows a computed tomography system according to an embodiment of the present invention.

### DETAILED DESCRIPTION

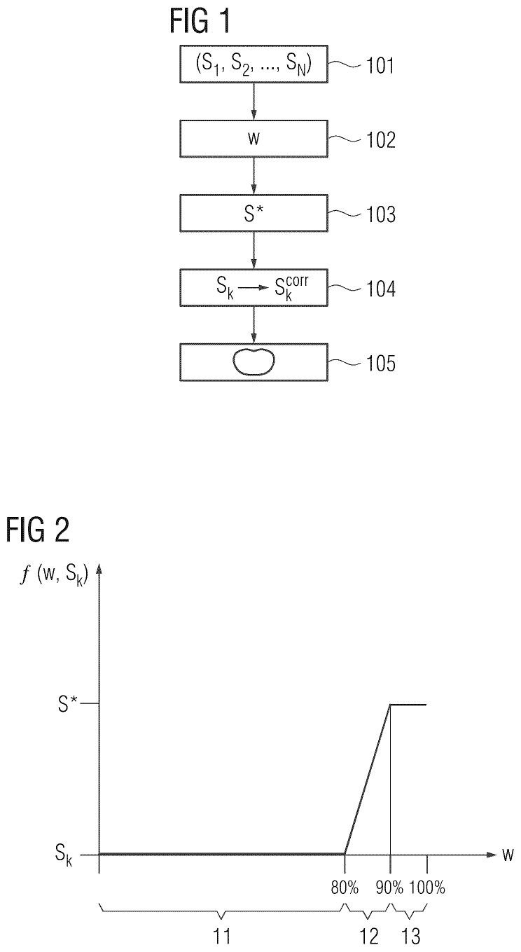

FIG. 1 shows a flow diagram of a method for the reduction of artifacts in spectral computed tomography image data according to an embodiment of the present invention. In a first step 101 computed tomography measurement data with sets of measurement values are received. One set {S₁, S₂, . . . , S_{N}} corresponds to one detector element readout of the measurement data and comprises measurement values Sₖ (k=1, 2, . . . . N) from N different x-ray spectra. N is at least two, hence, there are at least 2 two different x-ray spectra. In the case of exactly two different x-ray spectra per set, the set has the form {S₁, S₂}. This particular example of two different x-ray spectra per set may correspond to dual energy CT.

In a further step 102, an error estimate w is determined and/or received per set of measurement values. For example, the error estimate may be based on determining the error of data that is erroneous due to scattered radiation. An error estimate for scattered radiation may be determined, for example, via a maximum ratio between an estimated scatter signal Sₖ^{sc}≥0 and a total signal strength Sₖ of the respective measurement values. For example, such an error w may be determined according to the term

$$w = {max}_{k} min ( 1 , {S}_{k}^{s c} / {S}_{k} ) .$$

Additionally or alternatively, the error estimate w may be determined based on data errors in a low signal domain, such that a high error is assumed for low detector signals, such as based on parameters Sₖ^{L} and Sₖ^{H}, k=1, . . . , N defining the signal level range where measurements are to be considered as unreliable, e.g.:

$$w = {max}_{k} {cos}^{2} ( \frac{π}{2} max ( 0 , min ( 1 , \frac{{S}_{k} - {S}_{k}^{L}}{{S}_{k}^{H} - {S}_{k}^{L}} ) ) ) .$$

In a further step 103, for at least some of the sets, a replacement reference value S* per set of measurement values is defined. Optionally, the replacement reference value may be defined such that it is a linear combination of the measurement values of one set of measurement values, e.g. as a convex combination

S*=Σₖ₌₁^{N}cₖSₖ.

Preferably, the sum of coefficients cₖ of the linear combination is essentially 1, i.e., Σₖ₌₁^{N}cₖ=1. In on example, one of the coefficients cₖ is 1 and the other coefficients are zero. Hence, in this example, the replacement reference value is defined to be one of the measurement values of the respective set of measurement values.

In a further step 104, a function that maps the measurement values Sₖ to corrected values Sₖ^{corr} is applied. The corrected values Sₖ^{corr} are in between the mapped measurement value Sₖ and the replacement reference value S* of the respective set. The distance of the corrected value to each of S* and Sₖ depends on the error estimate w such that for larger errors the corrected value is closer to or at the replacement reference value S* and for smaller errors the corrected value Sₖ^{corr} is closer to or at the measurement value Sₖ. For example, a function ƒ(w; Sₖ), in particular monotonic function, may be used for mapping the measurement values Sₖ to the corrected values Sₖ^{corr}=ƒ(w; Sₖ). For example, assuming an error estimate ranging from 0 for no error to 1 for unsuitably large error, the function may yield ƒ(0; Sₖ)=Sₖ and ƒ(1; Sₖ)=S*. Optionally, the mapping may be defined according to multiple error ranges of the error estimate, such that for different error ranges a different partial mapping function is applied.

One example of a mapping function having different error ranges 11, 12, 13 is shown in FIG. 2. In the embodiment shown in FIG. 2, the error estimate ranges from 0% estimated error to 100% estimated error, where 100% error means the corresponding set of measurement values is completely unsuitable for spectral decomposition. While, in this representation, the replacement reference value is above the measurement value, it can in principle be higher or lower than the measurement value. The function ƒ(w; Sₖ) is separated into three ranges 11-13, a first range 11 below 80%, a second range 12 between 80% and 90%, and a third 13 range above 90%. In the first range 11, i.e. below an error estimate of 80%, the function maps the measurement value such that the corrected value is the measurement value Sₖ^{corr}=ƒ(w; Sₖ)=Sₖ. Hence, the measurement value is not changed in this first range 11. In the second range 12, i.e. between an error estimate of 80% and 90%, the function maps the measurement value such that the corrected value Sₖ^{corr} is between the measurement value Sₖ and the replacement reference value S*. Accordingly, in the second range 12, the measurement value is corrected to some degree. In the third range 13, i.e. above an error estimate of 90%, the function maps the measurement value such that the corrected value is the replacement reference value Sₖ^{corr}=ƒ(w; Sₖ)=S*. Accordingly, in the third range 13, measurement values are corrected maximally.

Referring back to FIG. 1, in a further step 105, computed tomography image data is reconstructed based on the corrected values.

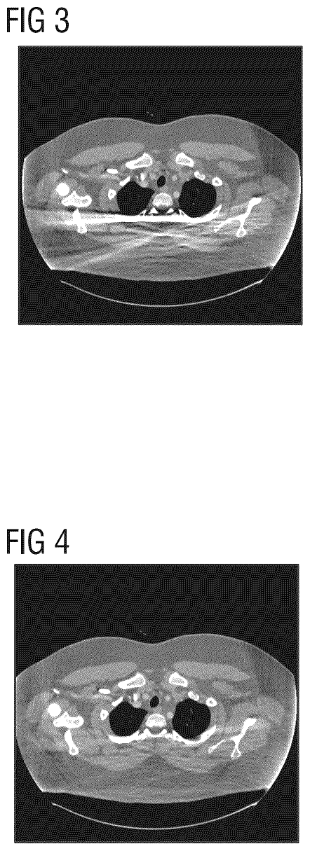

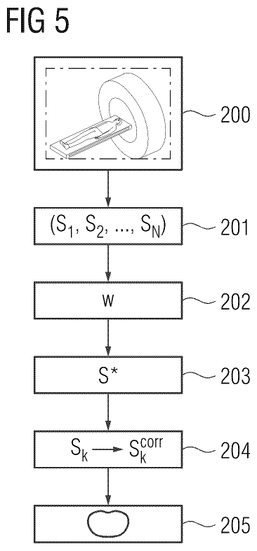

FIG. 3 shows a reconstructed spectral computed tomography image, where measurement values have not been corrected according to embodiments of the present invention. On the other hand, FIG. 4, shows a spectral computed tomography image, where corrected values that have been corrected according to embodiments of the present invention, have been used for the image reconstruction. In FIG. 3, depicting an axial CT image from a dual source thorax examination, one can clearly see several (white) streak artifacts passing about horizontally through one scapula, ribs, and vertebra. In FIG. 4, these artifacts have mostly been eliminated due to the application of a method according to embodiments of the present invention.

FIG. 5 shows a flow diagram of a method for providing spectral computed tomography image data according to an embodiment of the present invention. In a first step 200, measurement data are acquired by performing a spectral computed tomography scan. The following steps 201-205 may be applied equivalently to the steps 101-105 as described with respect to FIG. 1.

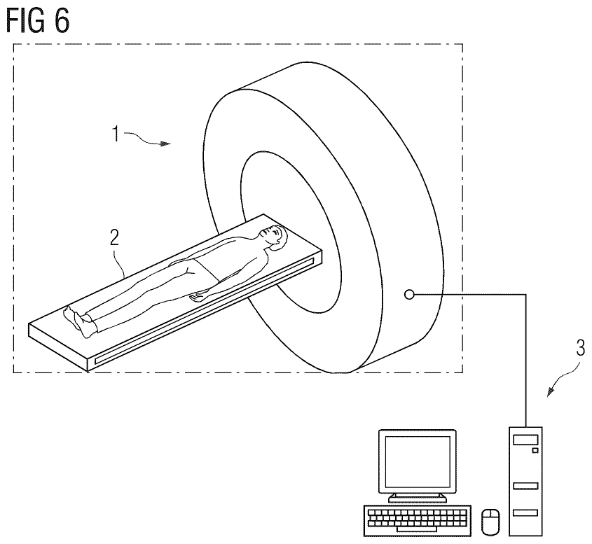

FIG. 6 shows a computed tomography system according to an embodiment of the present invention. In this embodiment, the computed tomography system comprises a gantry 1, a movable patient table 2 and a processing unit 3 that is configured to carry out a method as described herein, for example, as described with respect to FIG. 5.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

What is claimed is:

1. A computer-implemented method for reduction of artifacts in spectral computed tomography image data, the computer-implemented method comprising:
receiving computed tomography measurement data with sets of measurement values, wherein each set of measurement values corresponds to a respective detector element readout and includes measurement values for at least two different x-ray spectra;
at least one of determining or receiving an error estimate for each set of measurement values;
defining a replacement reference value for each set of measurement values;
applying a function that maps measurement values to corrected values, wherein
for each respective measurement value, a respective corrected value is between the respective measurement value and the replacement reference value for the set of measurement values including the respective measurement value, and
a distance of the respective corrected value to each respective measurement value depends on the error estimate such that for larger errors the respective corrected value is closer to or at the replacement reference value for the set of measurement values including the respective measurement value, and for smaller errors the respective corrected value is closer to or at the respective measurement value; and
reconstructing computed tomography image data based on the corrected values.

2. The computer-implemented method according to claim 1, wherein the replacement reference value for the set of measurement values including the respective measurement value is in an order of magnitude of the measurement values of the set of measurement values including the respective measurement value.

3. The computer-implemented method according to claim 1, wherein the replacement reference value for the set of measurement values including the respective measurement value is defined such that the replacement reference value is a linear combination of the measurement values of the set of measurement values including the respective measurement value.

4. The computer-implemented method according to claim 3, wherein at least one of coefficients of the linear combination are the same within each mapped set, or a sum of the coefficients of the linear combination is 1 or substantially 1.

5. The computer-implemented method according to claim 1, wherein the replacement reference value for the set of measurement values including the respective measurement value is defined to be one of the measurement values in the set of measurement values including the respective measurement value.

6. The computer-implemented method according to claim 1, wherein the error estimate is based on determining an error of data that is erroneous due to scattered radiation.

7. The computer-implemented method according to claim 6, wherein the error estimate is determined based on a ratio of an estimated scatter signal to a total signal strength of the measurement values of the set of measurement values including the respective measurement value.

8. The computer-implemented method according to claim 1, wherein the error estimate is determined based on data errors in a low signal domain, such that a high error is assumed for low detector signals.

9. The computer-implemented method according to claim 1, wherein the function that maps the measurement values to the corrected values is a monotonic function.

10. The computer-implemented method according to claim 1, wherein the mapping is defined according to at least two different error ranges of the error estimate, such that for each error range a different partial mapping function is applied.

11. The computer-implemented method according to claim 1, wherein
the error estimate includes at least three error ranges according to which the mapping is applied, and
the error ranges include
a first error range of low errors for which the respective corrected value is set to the respective measurement value,
a second error range of medium errors for which the measurement values are mapped such that the respective corrected value is between the respective measurement value and the replacement reference value for the set of measurement values including the respective measurement value, and
a third error range of high errors for which the respective corrected value is set to be the replacement reference value for the set of measurement values including the respective measurement value.

12. A method for providing spectral computed tomography image data, the method comprising:
acquiring measurement data by performing a spectral computed tomography scan; and
performing the computer-implemented method according to claim 1.

13. A non-transitory computer-readable storage medium storing computer-executable instructions that, when executed by a computer, cause the computer to carry out the computer-implemented method of claim 1.

14. A computed tomography system comprising a processing unit configured to carry out the computer-implemented method according to claim 1.

15. The computer-implemented method according to claim 7, wherein the ratio is a maximum ratio between the estimated scatter signal and the total signal strength.

16. The computer-implemented method according to claim 2, wherein the replacement reference value for the set of measurement values including the respective measurement value is defined such that the replacement reference value is a linear combination of the measurement values of the set of measurement values including the respective measurement value.

17. The computer-implemented method according to claim 4, wherein the replacement reference value for the set of measurement values including the respective measurement value is defined to be one of the measurement values of the set of measurement values including the respective measurement value.

18. The computer-implemented method according to claim 7, wherein the mapping is defined according to at least two different error ranges of the error estimate, such that for each error range a different partial mapping function is applied.

19. The computer-implemented method according to claim 7, wherein
the error estimate includes at least three error ranges according to which the mapping is applied, and
the error ranges include
a first error range of low errors for which the respective corrected value is set to the respective measurement value,
a second error range of medium errors for which the measurement values are mapped such that the respective corrected value is between the respective measurement value and the replacement reference value for the set of measurement values including the respective measurement value, and
a third error range of high errors for which the respective corrected value is set to be the replacement reference value for the set of measurement values including the respective measurement value.

20. A computed-tomography system for reduction of artifacts in spectral computed tomography image data, the computed-tomography system comprising:
a memory storing computer-readable instructions; and
at least one processor configured to execute the computer-readable instructions to cause the computed-tomography system to
receive computed tomography measurement data with sets of measurement values, wherein each set of measurement values corresponds to a respective detector element readout and includes measurement values for at least two different x-ray spectra,
at least one of determine or receive an error estimate for each set of measurement values,
define a replacement reference value for each set of measurement values,
apply a function that maps measurement values to corrected values, wherein
for each respective measurement value, a respective corrected value is between the respective measurement value and the replacement reference value for the set of measurement values including the respective measurement value, and
a distance of the respective corrected value to each respective measurement value depends on the error estimate such that for larger errors the respective corrected value is closer to or at the replacement reference value for the set of measurement values including the respective measurement value, and for smaller errors the respective corrected value is closer to or at the respective measurement value, and
reconstruct computed tomography image data based on the corrected values.
